(19) United States
(12) Patent Application Publication
(10) Pub. No.: US 2003/0232377 A1
(43) Pub. Date: Dec. 18, 2003

(54) Early noninvasive prenatal test for aneuploidies and heritable conditions

(75) Inventor: John O. Thomas, Brooklyn, NY (US)

Correspondence Address: BROWDY AND NEIMARK, P.L.L.C., 624 NINTH STREET, NW, SUITE 300, WASHINGTON, DC, 20001-5303, US

(73) Assignee: New York University, New York, NY (US)

(21) Appl. No.: 10/459,557

(22) Filed: Jun. 12, 2003

Related U.S. Application Data
(60) Provisional application No. 60/387,914, filed on Jun. 13, 2002.

(51) Int. Cl. C12Q 1/68; C12N 5/08

(52) U.S. Cl. 435/6; 435/372

(57) ABSTRACT

The present invention provides methods for isolating fetal cells from samples of maternal blood, and for detecting aneuploidies and heritable disorders. Fetal cells are enriched from maternal blood samples using layered immunosorption to specifically bind erythroid cell precursors. In the layered immunosorption method, a substrate such as a microscope slide is coated with a thin layer of erythrocyte membranes. Prior to assay the membranes are activated by binding an antibody against an erythroid cell surface protein. The sample is then added to the substrate and incubated. Nonadsorbed cells are removed by washing, and the bound erythroid cells are permanently attached by fixation and drying. Molecular beacons or other molecular probes are used for differentially detecting fetal and maternal cells. Permeabilizing detergents are used for purifying and detecting fetal cells.

### CROSS-REFERENCE TO RELATED APPLICATION

The present invention is the non-provisional of Serial No. 60/387,914, filed Jun. 13, 2002, the entire contents of which are hereby incorporated by reference.

### FIELD OF THE INVENTION

The present invention relates to a method for noninvasive early detection of chromosomal aneuploidies based on fetal cells present in peripheral maternal blood as well as to a method for isolating fetal cells from maternal blood.

### BACKGROUND OF THE INVENTION

Chromosomal abnormalities occur in 0.1% to 0.2% of live births. Among these, the most common clinically significant abnormality is Down syndrome (trisomy 21). Currently there are both diagnostic and screening tests for chromosomal abnormalities, but, unfortunately, all of them have serious limitations. The diagnostic tests involve small but significant risks to the fetus and mother, and the screening tests suffer from less than desirable sensitivity and/or specificity. Because of these limitations, a great deal of effort is currently being directed toward the development of improved screening and diagnostic tests. One approach that is being explored in several laboratories is to isolate fetal cells from the mother's blood, and to use the DNA from these cells for prenatal diagnosis. Such a test would have two compelling advantages over those that are currently available: it would be noninvasive, and it could be done very early in pregnancy. The major problem that must be overcome for this approach to be feasible is how to isolate the fetal cells, which are present in the mother's blood in very small numbers.

The diagnostic test for chromosomal abnormalities, including trisomy 13, trisomy 18, Klinefelter syndrome, XYY, Turner syndrome, and Down syndrome, is the cytogenetic analysis. This is a highly accurate and well-established test, but there is major disadvantage in that the test requires an invasive procedure, either amniocentesis or chrorionic villus sampling (CVS) to obtain fetal tissue. This presents three problems: (1) risk to both the fetus and the mother, (2) a delay in diagnosis, and (3) cost. Because amniocentesis and CVS are invasive procedures, there is a small but significant risk to the fetus and a slight risk of infection for the mother. Amniocentesis is generally done at 15 weeks of gestation, although at some centers it is performed as early as 11-14 weeks. Chorionic villus sampling is done at 9-12 weeks gestation. The earlier diagnosis afforded by CVS or early amniocentesis is advantageous because of reduced emotional stress on the parents, and from the medical advantages associated with an early termination of pregnancy if that is what the parents choose. However, the earlier diagnosis entails an increased risk to the fetus.

The risk of fetal loss is small but significant. It is generally quoted that there is about a 0.5% risk of fetal loss as a consequence of a mid-trimester (16 week) amniocentesis, although the actual risk is probably lower than this. The risk associated with early amniocentesis (14 weeks) or CVS is somewhat greater (Johnson et al., 1999; Sundberg et al., 1997; Wilson, 2000). For women under age 35 without a predisposing factor, the risk of fetal loss due to amniocentesis is greater than the incidence of Down syndrome. Hence, the diagnostic test is generally recommended only for women at age 35 or over unless there is another predisposing factor. The most common predisposing factor is a positive screening test. For women over 35, the incidence of Down syndrome increases rapidly with increasing age. At age 35 the incidence is about 1/200 live births, and increases to about 1/46 at age 45. Although the risk of Down syndrome (as well as other chromosome abnormalities) is greatly increased, the consequences of a fetal loss due to amniocentesis are also much greater, since these women may not be able to achieve another pregnancy.

Because of the risks associated with the prenatal diagnostic tests currently available, a large amount of effort has been dedicated towards developing screening tests. Whereas the diagnostic test is a highly accurate and sensitive way of detecting chromosomal aneuploidies, the screening tests that are currently available provide only an indication of whether or not the fetus is affected with Down syndrome. A negative result from a screening test does not mean that the child will be unaffected, and a positive result must be followed up by the diagnostic test to be meaningful. Because of the relatively low specificity of the current screening tests and the requirement that positive tests be validated by the diagnostic cytogenetic test, a large number of normal pregnancies are jeopardized by amniocentesis.

Currently, there are two types of screening tests available: a blood test conducted on the mother, and an ultrasound test conducted on the fetus. The blood test is done in the second trimester, typically between 15 and 20 weeks gestation. In this test, a blood sample is taken from the mother and the levels, of one, two, three, or four biochemical markers are determined. This test is referred to as a “triple screen” if three markers are determined, or a “quad screen” if four markers are determined. The results of these tests also serve as a screening test for trisomy 18 and for neural tube defects.

The use of a triple screen for pregnant women under age 35 is currently the standard of practice and is covered by most insurance companies. The markers that are measured in the triple screen are alpha-fetoprotein, chorionic gonadotropin, and unconjugated estriol. Recently, a fourth biochemical marker, inhibin-A, has been added to the triple screen to form the “quad screen.”

Since the triple screen has been in use for a number of years, a considerable amount of data on the sensitivity and specificity of the test has been accumulated (Hjudered-Duric et al., 2000; McDuffie et al., 1996; Spencer, 1999; Tanski et al., 1999). The sensitivity and specificity vary with the age of the mother and with the cutoff criteria used by various investigators, but is generally quoted as follows. Out of 1000 women tested, about 100 will test positive with a recommendation to follow up with amniocentesis for a cytogenetic study. Of these, two or three will actually have a fetus with Down syndrome. Of those who test negative, two will have a child with Down syndrome. Thus, many providers do not like this test, since it does not provide the parents with greatly increased assurance of a child without Down syndrome, subjects many couples to the emotional effects associated with receiving a positive test, and subjects many normal fetuses to the risks of amniocentesis.

Second-trimester ultrasound screening is a routine part of prenatal care in many practices, and several sonographic markers have been associated with chromosomal abnormalities. In a recent article (Smith-Bindman et al., 2001), studies conducted between 1980 and 1999 were reviewed to determine the accuracy with which each of these markers was able to detect Down syndrome. The authors found that in the absence of associated fetal abnormalities, the sensitivity of any of these markers was low (1% to 16%). Because of the relatively low sensitivity and relatively high false positive rate, the authors concluded, “Using these markers as a basis for deciding to offer amniocentesis will result in more fetal losses than cases of Down syndrome detected, and will lead to a decrease in the prenatal detection of fetuses with Down syndrome.”

For over a decade it has been realized that fetal cells are present in the mother's blood, and that these cells present a potential source of fetal chromosomes for prenatal DNA-based diagnostics. Since these cells appear very early in the pregnancy, they could, in principle, form the basis of an accurate noninvasive first trimester test (Lamvu and Kuller, 1997; Lim et al., 2001; Shulman et al., 1998). The difficulty with this approach is that there are very few fetal cells, on the order of about 1 per milliliter, although there are some data indicating that in aneuploid pregnancies there may be considerably more fetal cells present in the maternal circulation (Zhong et al., 2000). Over the past few years, a number of methods for isolating these cells have been proposed, and a multi-center trial (NYFTI) (Bianchi et al., 1999) is in progress to evaluate the clinical feasibility of one of these approaches.

In addition to fetal cells, it is also now clear that there is a considerable amount of fetal DNA present in the maternal circulation (Bischoff et al., 1999; Lo, 2000). For diagnosing aneuploidies such as Down syndrome, however, cells are a preferred source of material.

The approaches for isolating fetal cells from maternal blood that have been proposed to date entail combinations of the following enrichment, amplification, and identification steps:

1. Removal of red blood cells by density gradient centrifugation or preferential cell lysis;

2. Amplification by cell culture methods;

3. Enrichment by cell sorting;

4. Identification by immunological methods.

Once the cells are isolated, genetic analysis is by standard methods, either interphase fluorescence in situ hybridization (FISH) for determining aneuploidies, or by polymerase chain reaction (PCR) for other conditions that may be indicated in a particular pregnancy. While a number of these approaches have been demonstrated to work in a laboratory setting, none has reached a level of development to be considered for routine clinical use. Perhaps the furthest developed protocol is the one developed by Bianchi's lab, described below, which is currently being evaluated in a multi-center trial

The following is a brief overview of the enrichment, amplification, and identification steps that are currently under consideration. Most procedures start with eliminating red blood cells by density gradient centrifugation, either through hypaque or percol gradients (DiNaro et al., 2000; Samura et al., 2000; Smits et al., 2000; Sezikawa et al., 2000). This is a standard hematological protocol modified slightly to either collect all nucleated cells or to preferentially enrich for nucleated erythroid cells. An alternative method is to dilute the blood into a buffer that lyses mature red blood cells but not nucleated cells (Huber et al., 2000). Whichever procedure is used, the result is a population of nucleated cells from the mother that contains a very small number of fetal cells.

Some investigators have suggested that the small number of fetal cells can be preferentially increased at this stage by culturing under conditions that favor the growth of fetal erythroid cells (Han et al., 2001; Huber et al., 2000; Tutschek et al., 2000). The results of these studies have, however, been controversial (Jansen et al., 2000). Most of the studies that have demonstrated a sizeable amplification when done with synthetic mixtures of fetal cord blood and adult blood do not work on the fetal cells present in the maternal circulation. If conditions for the preferential amplification of fetal cells are found, they will be a valuable addition for almost any protocol. The additional time required for amplification would be more than offset by the fact that cells could be obtained early in the pregnancy.

The crucial stage in most protocols is the separation of fetal cells from the vast excess of nucleated maternal cells. Most approaches for doing this rely on some form of cell sorting, most commonly either fluorescence activated cell sorting (FACS) and/or magnetic activated cell sorting (MACS). To accomplish the cell sorting, the fetal cells must be labeled, most commonly with an antibody to a particular cell protein that is preferentially expressed by fetal cells. Several targets for labeling have been proposed. In the procedure used by Bianchi's group and in the NIFTY trial (Sekizawa et al., 2000), the cells are labeled with fluorescent antibodies against fetal hemoglobin following fixation and permeabilization of the cells. The labeled cells are then sorted by FACS. Other investigators have used antibodies to other hemoglobin subunits (Al-Mufti et al., 2001; DiNaro et al., 2000) or cell surface antigens such as CD34, CD71 (transferring receptor), glycophorin A, CD36 (thrombospondin receptor) (Bischoff et al., 1998; Elias et al., 1996; Rodriguez De Alba et al., 2001; Smits et al., 2000; Wang et al., 2000). It is also possible to enrich for fetal cells by eliminating cells that express CD45, a protein that is present on the surface of lymphocytes, but not red blood cell precursors. This is usually done by magnetic cell sorting methods.

Following enrichment, the cells are mounted on a microscope slide by standard cytological methods for chromosomal analysis by FISH. In many protocols the cells are also stained for fetal hemoglobin to further distinguish fetal cells from contaminating maternal cells. Several methods have been used for this step. The most widely used is to stain the cells with a fluorescent antibody to the gamma globin chain. Fetal cells express the gamma chain of hemoglobin, whereas most maternal cells express the beta chain of hemoglobin. Other probes, such as antibodies to the zeta chain of hemoglobin, and a chemical staining method adapted from the Kleinhaur test, have also been suggested (Martel-Petit et al., 2001).

Although there are multiple criteria for distinguishing fetal from maternal cells, they must be used with care, since each step in an enrichment scheme entails the loss of precious cells. Although it is difficult to objectively evaluate the relative yields of all of the different procedures, it has been shown that simple protocols are superior to more complex ones.

A typical protocol for isolating fetal erythroid cells from the maternal circulation is the one developed by Bianchi's lab (Samura et al., 2000). This procedure entails:

1. Isolating mononuclear cells by density gradient centrifugation onto Histopaque at a density of 1.09 g/ml;

2. Depleting lymphocytes and monocytes by labeling them with anti-CD45 and separating with magnetic activated cell sorting;

3. Fixation with paraformaldehyde and permeabilization with methanol:acetone;

4. Labeling with fluorescent anti-gamma globin and the dye Hoechst 3342 which stains nuclei;

5. Selecting positively staining cells by fluorescent activated cell sorting (FACS);

6. Attaching the sorted cells to a microscope slide;

7. Hybridizing the cell's DNA with fluorescence in situ hybridization (FISH) probes;

8. Selecting cells exhibiting cytoplasmic fluorescence (those cells containing gamma-globin, presumably fetal erythroid cells), and observing the FISH staining patterns of those cells.

In at least some circumstances the mother can retain fetally derived cells for many years following a pregnancy (Bianchi, 2000). Presumably these are derived from fetal stem cells that take up residence in various tissues of the mother where they give rise to differentiated cell types. It is therefore necessary to consider this potential source of fetal cells in the test design. In the tests proposed here, only gamma globin producing cells are detected, and it is highly unlikely that fetally derived cells from a previous pregnancy would produce gamma globin. The test proposed by Bianchi's group also only detect gamma globin producing cells.

### SUMMARY OF THE INVENTION

It is an object of the present invention to overcome the aforesaid deficiencies in the prior art.

It is another object of the present invention to provide a method for early, noninvasive, detection of Down syndrome and other aneuploidies.

It is still another object of the present invention to provide a method for early detection of heritable conditions other than aneuploidies.

It is a further object of the present invention to provide a layered immunosorption method for the isolation, purification, and identification of fetal cells.

It is another object of the present invention to apply specific molecular beacons for differentially detecting fetal and maternal cells.

It is yet another object of the present invention to use permeabilizing detergents in purifying and detecting fetal cells.

The present invention provides a protocol to select and identify fetal erythroid cells by minimizing manipulations of cells and hence minimizing the loss of rare fetal cells, to be low tech and hence minimize costs, and to be rapid, minimizing costs and increasing throughput.

The basis of the procedure of the present invention is a layered immunosorption step to isolate nucleated erythroid cells. Previous estimates suggest that about ⅓ of these cells are fetal in origin. This step is followed by differentially detecting fetal vs. maternal cells through the use of molecular probes designed to specifically recognize either proteins or RNAs expressed specifically or preferentially by fetal cells.

### BRIEF DESCRIPTION OF THE DRAWINGS

FIG. 1 shows layered immunosorption of erythrocytes onto a microscope slide.

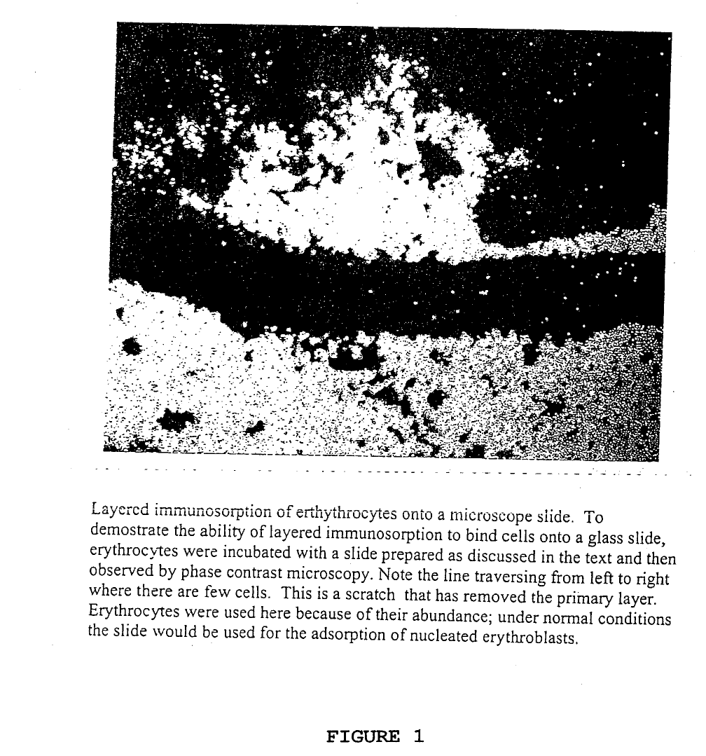

FIGS. 2A, 2B, and 2C show fetal cells detected from a 1:10,000 mixture of fetal and adult blood. The blood samples were observed by phase contrast microscopy (FIG. 2A), Hoechst 332258 staining for nuclei (FIG. 2B), and molecular beacon stating for gamma-globin mRNA.

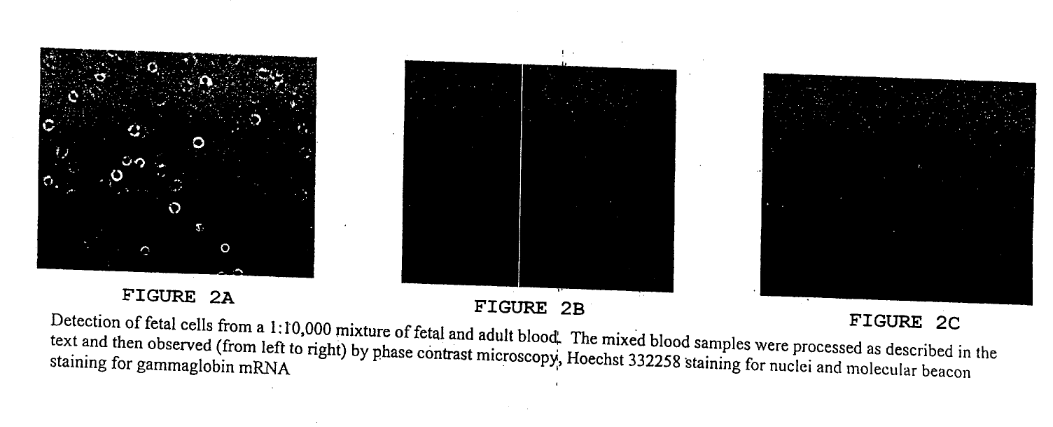

### DETAILED DESCRIPTION OF THE INVENTION

The present invention provides two related approaches to isolating fetal cells: one for early detection of Down syndrome and other aneuploidies, and one for early detection of other heritable conditions. These approaches rely on three innovations:

1. Development of a layered immunosorption method;

2. Application of specific molecular beacons for differential detection of maternal and fetal cells; and

3. Use of permeabilizing detergents in the purification and detection of fetal cells.

Detection of Down Syndrome and Other Aneuploidies

The method of the present invention has been designed to minimize manipulations of the cells and hence minimize the loss of rare fetal cells, to be low tech and hence minimize costs, and to be rapid, minimizing costs and increasing throughput. The basis of the procedure is a layered immunosorption step to isolate nucleated erythroid cells, about ⅓ of which are fetal in origin. This step is followed by differential detection of fetal vs. maternal cells through the use of molecular probes designed to specifically recognize either proteins or RNAs expressed specifically or preferentially by fetal cells.

A sample of maternal blood collected by venipuncture into EDTA tubes is centrifuged through a density gradient so as to separate nucleated cells from erythrocytes. Such a gradient might consist of the blood sample layered over a solution of histopaque® with a density of 1.19 g/ml. The mononuclear cells present at the density interface are harvested and then adsorbed onto a specifically treated microscope slide designed to retain erythroid cells, both maternal and fetal. After fixation in a fixative such as formaldehyde and a brief wash, the slide with attached cells is dried, and processed for FISH according to a standard interphase FISH protocol. The cells are stained, either before or after the FISH procedure with a molecular probe designed to recognize proteins or RNAs that are specifically or preferentially expressed by fetal cells. Such a probe may be a fluorescently labeled antibody to a fetal or embryonic hemoglobin, or a molecular beacon complementary to a fetal or embryonic globin mRNA, or a fluorescently labeled oligonucleotide complementary to a fetal or embryonic globin mRNA. When observed by fluorescence microscopy, fetal cells can be distinguished from maternal cells by color, and the hybridization pattern of the FISH probes associated with fetal cells can be determined.

Detection of Other Heritable Conditions

A maternal blood sample collected by venipuncture into EDTA tubes is diluted into phosphate buffered saline to give a final solution with a set number of red blood cells/ml. A carefully controlled amount of a permeabilizing detergent, such as lysolecithin, digitonin, NP40, triton X100 is added together with a fluorescein-labelled molecular beacon specific for gamma-, zeta-, or epsilon-globin mRNA and a rhodamine labeled molecular beacon specific for beta-globin mRNA. The permeabilizing detergent does two things: it lyses the red blood cells, and also partially permeabilizes the nucleated cells, permitting entry of the molecular beacons, which hybridize with their specific target mRNAs. Any combination of fluorescent labels can be used so long as the two labels make it possible to distinguish maternal cells from fetal cells. Other types of labels can be used to distinguish maternal cells from fetal cells, such as those described in Bianchi, U.S. Pat. No. 5,641,628 and U.S. Published Application 2002/0006621, the entire contents of which are hereby incorporated by reference.

Once labeled, the cells can then either be sorted by FACS or enriched by removal of CD-45 positive cell and then sorted by FACS. The purity of the resulting preparation can be monitored by fluorescent microscopy, and the fetal cells used for analysis by PCR amplification. The use of molecular beacons has several advantages over technologies that use antibodies against cell surface markers or globin proteins:

1. The use of fetal hemoglobin mRNA as a marker is much more specific than the use of surface antigens;

2. The small size of the molecular beacons makes it possible to use partially permeabilized cells;

3. Under stringent conditions of hybridization, the molecular beacon is highly specific;

4. The molecular beacon produces very little background fluorescence in the absence of fetal globin mRNA, so that there is no need for extensive washing of the cells;

5. Labeling the cells is simple, involving the addition of one solution and a ten-minute incubation;

6. Molecular beacons are much less expensive than antibody-based technologies.

Layered Immunosorption

The method of layered immunosorption offers significant advantages over existing methods for enriching fetal cells or, in principle, any other cell type. It requires no expensive equipment, it is rapid, and it can be easily scaled up for large-scale screening operations. While any suitable substrate can be used, a microscopic slide is preferred.

A microscope slide is prepared by coating a small portion of the slide with a thin layer of erythrocyte membranes. The coated slides are stable and thus can be prepared in advance. Prior to the assay, the membranes are activated by treatment with antibodies that specifically or preferentially recognize erythroid cell surface proteins. Examples of such antibodies are monoclonal antibodies to glycophorin A and/or transferrin receptor. The slides are then used for selecting erythroid cells by simply adding a solution of mononuclear cells to the slide, which can be facilitated by a well attached to the slide, and allowing the cells to settle and attach to the slide. Nonabsorbed cells are removed by a brief gentle wash, and the erythroid cells are permanently fixed to the slide by drying and methanol/acetic acid fixation.

The substrate for use in the layered immunosorption is coated with any coating that will bind to a protein present on the surface of fetal cells. One skilled in the art can readily determine which coatings are appropriate for this process by determining which compounds bind to a protein present on fetal cells, without undue experimentation. Examples of suitable coatings include a layer of erythrocyte membranes bound to the surface of the substrate and activated by treatment of antibodies that react with an erythroid cell surface protein. Examples of such cell surface proteins include glycophorin A, glycophorin B, and transferrin receptors.

Antibodies can be attached to the substrate for selecting erythroid cells by any suitable method. These methods include direct chemical attachment of the antibodies to the microscope side, and indirect methods for attaching antibodies, such as using S. aureus protein A.

Differential Detection with Molecular Beacons or Oligonucleotide Probes

All current technologies for detecting fetal cells use monoclonal antibodies directed against markers specific to fetal cells. The most widely used are anti-gamma globin antibodies. In contrast thereto, the present invention uses oligonucleotides, specifically molecular beacons, directed against globin mRNA. Molecular beacons are oligonucleotides that fluoresce only when bound to the target DNA or RNA sequence (Bonner et al., 1999). Molecular beacons are designed to form a hairpin loop with a fluorescent group at one end and a group that quenches fluorescence at the other end. The hairpin loop brings the two groups together, so that fluorescence is quenched. When bound to a target DNA or RNA, the ends are separated, and the molecule fluoresces.

Molecular beacons are well suited for differential detection of maternal and fetal cells because they are easy to use: all that is required is to include them in the final mounting solution, and there is no requirement for washing. This is particularly important when using PCR detection of heritable disorders. In this case, the probe is added to permeabilized cells, where washing would be difficult, since the cells do not stay permeabilized for long. This is a lesser consideration for detecting aneuploidies, where a brief wash would be acceptable. Molecular beacons are inexpensive, since they both cost less than antibodies and are much easier to use. The detection signal is reversible.

It may also be possible to design oligonucleotide probes, not necessarily molecular beacons, that dissociate from the target mRNA at a low temperature relative to FISH probes. The advantage of this is that it may permit the use of an additional FISH probe with the same color as the fetal cell detection probe. Since oligonucleotide probes are highly specific, two probes can be used to further increase the ability to distinguish fetal and adult cells. One example of this is a green probe for adult beta globin mRNA and a red probe for fetal gamma- and/or epsilon- and/or zeta-mRNA. This combination of colors is particularly useful in cases such as a hereditary persistence of fetal hemoglobin or thalssemia, where adult cells express a combination of fetal and adult globin mRNAs. In these cases, some red color will be present, but the cells would be clearly counted as adult cells. This is in contrast to tests that rely on anti-gamma-globin antibodies, where these adult cells appear weakly positive, and thus may be confused with fetal cells. Another advantage is that the intensity of the signal can be greatly increased by using multiple probes for each mRNA. However, it has been found that the signals are quite intense using only one probe.

Any molecular suitable probes can be used in the present invention. Example of molecular probes can be found in Coull et al., U.S. Pat. No. 6,355,421, the entire contents of which are hereby incorporated by reference.

Cell Permeabilization

Detergents such as lysolecithin or digitonin are able to minimally permeabilize cells. Under appropriate conditions, nucleated cells remain largely intact but become permeable to oligonucleotide-probe sized molecules (Li and Thomas, 1989). Further, red blood cells are much more sensitive to permeabilization than nucleated cells, so that it is possible to eliminate red cells by lysis rather than by density gradient centrifugation. This is advantageous, since significant cells losses accompany the centrifugation step. Hube et al. (2000) have also used preferential lysis rather than density gradient centrifugation as an initial step. However, they achieved lysis by diluting the blood sample into a hypotonic buffer. In the present invention, it has been discovered that it is essential to carefully adjust the cell density prior to permeabilization, presumably because the cells bind detergent and therefore reduce the free concentration of the detergent.

Detection of Down Syndrome and Other Aneuploidies

The method of the present invention has been demonstrated to work with mixtures of fetal and adult blood. The fetal blood was obtained, with institutional review board approval, from discarded umbilical cords immediately following delivery, and the adult blood was obtained from a donor. Experiments in which known numbers of fetal cells (tens of cells) are added to adult blood, suggest that a yield of about 90% is obtained with the described procedure.

The layered immunosorption of the present invention has been found to be highly effective for isolating erythroid precursor cells. A substrate, such as a glass microscope slide, is coated with a thin layer of erythrocyte ghosts, which is then activated with antibodies against erythroid cell surface proteins. Although any type of substrate can be used for the layered immunosorption, it has been found that standard microscope slides are preferred. Microscope slides are very flat, the layers and the cells adhere well, and they have been proven to be effective in all procedures associated with FISH analysis.

Glycophorins are a predominant antigen on the erythrocyte surface, and anti-glycophorin antibodies are one embodiment of the present invention. However, anti-transferrin receptor antibodies or antibodies against any erythroid call surface protein can also be used, either alone or in conjunction with anti-glycophorin A and/or glycophorin B. Other workers have used anti-glycophorin A antibodies and FACS sorting to isolate fetal erythroblasts, but have encountered substantial problems with cell clumping due to the abundance of the protein. The layered immunosorption method of the present invention avoids clumping because the antibodies are bound to a surface. An antibody concentration of about 5 ng/mm² appears to be optimal. Greater amounts of antibody neither help greatly nor do they hinder the observation of the cells.

Cells are adsorbed onto the slide by applying the cells to a well attached to the slide and allowing the cells to settle onto the surface. Even though red blood cells adhere very well to the slide, contamination of the cell sample with a few red blood cells is not a problem. When the red blood cells are permeabilized in subsequent steps, these cells simply blend into the background of the erythrocyte ghost layer. A large contamination, however, is not desirable, since it would decrease space available for adsorption of fetal cells.

Following adsorption, the cells are fixed to the slide. This can be achieved by treatment with a fixative such as formaldehyde, Zanboni's fixative, Bouin's fixative, methanol/acetic acid, ethanol, or others. The samples are further fixed and bonded to the slide by drying.

In the process of the present invention, molecular beacons are used for differential detection of fetal and maternal cells. The most desirable probe is that for fetal gamma globin, which has very high sensitivity and specificity. Additionally, probes for epsilon goblin and zeta globin mRNA are also useful in this procedure.

After fixation, the cells are ready for analysis by FISH. Standard FISH methods are used.

The procedures of the present invention will be particularly useful as a screening test for Down syndrome and other common aneuploidies, either in place of or in conjunction with other screening tests that are currently under analysis, such as sonography and biochemical marker tests. For most women, knowing the status of the child for these common conditions is of great importance, either for peace of mind if the child is unaffected, emotional and medical preparation if the child is affected, or possible termination of the pregnancy. Because the test described here is noninvasive, it will potentially save many children who are currently lost due to the small but significant risk of amniocentesis.

The test of the present invention is equally effective for pregnant women of all ages. This is a significant point, since the current standard of care is to offer amniocentesis and cytogenetic testing only to women over age 35. Unfortunately, these women are those who are most sensitive to the risk of amniocentesis.

An early test is important. A negative test obtained early, as opposed to late in the pregnancy, would clearly have greater emotional value. A positive test would allow the couple greater time to consider options such as termination of the pregnancy, or for early amniocentesis or CVS for a follow up diagnostic test and, if so desired, an earlier termination of the pregnancy. The test of the present invention is administered in the first trimester of pregnancy, possibly as early as six weeks from conception.

In contrast to other methods for isolating fetal cells, the method of the present invention is both rapid and technologically simple. Currently, a major expense is in the cost of the FISH reagents. While the cost of the test of the present invention would clearly be greater than that of the triple screen, much of the increased expense will be countered by a lower false positive rate and hence a lowered demand for amniocentesis. For older women, the availability of a test such as the one of the present invention is a highly cost effective alternative to routine amniocentesis and cytogenetic testing. This will potentially save may children who are currently lost as a consequence of amniocentesis.

The foregoing description of the specific embodiments will so fully reveal the general nature of the invention that others can, by applying current knowledge, readily modify and/or adapt for various applications such specific embodiments without departing from the generic concept, and, therefore, such adaptions and modifications should and are intended to be comprehended within the meaning and range of equivalents of the disclosed embodiments. It is to be understood that the phraseology or terminology employed herein is for the purpose of description and not of limitation.

### References

Al-Mufti, R., et al. (2001) Distribution of fetal and embryonic hemoglobins in fetal erythroblasts enriched from maternal blood. Haematologica. 86:357-362

Benn, P. A., et al. (2001) Estimates for the sensitivity and false-positive rates for second trimester serum screening for Down syndrome and trisomy 18 with adjustment for cross-identification and double-positive results. Prenat Diagn. 21:46-51

Bianchi D. W. (200) Fetomaternal cell trafficking: a new cause of disease? Am J Med Genet. 91:22-8

Bianchi D. W., et al. (1999) Fetal cells in maternal blood: NIFTY clinical trial interim analysis. DM-STAT. NICHD fetal cell study (NIFTY) group. Prenat Diagn. 19:994-5

Bischoff F. Z., et al. (1999) Noninvasive determination of fetal RhD status using fetal DNA in maternal serum and PCR J Soc Gynecol Investig. 6:64-9

Bischoff F. Z., et al. (1998) Prenatal diagnosis with use of fetal cells isolated from maternal blood: five-color fluorescent in situ hybridization analysis on flow-sorted cells for chromosomes X, Y, 13, 18 and . . . Am J Obstet Gynecol. 179:203-9

Bonnet G., et al. (1999) Thermodynamic basis of the enhanced specificity of structured DNA probes. Proc Natl Acad Sci USA. 96:6171-6

Di Naro E., et al. (2000) Prental diagnosis of beta-thalassaemia using fetal erythroblasts enriched from maternal blood by a novel gradient. Mol Hum Reprod. 6:571-4

Elias S., et al. (1996) Isolation and genetic analysis of fetal nucleated red blood cells from maternal blood: the Baylor College of Medicine experience. Early Human Dev. 47 Suppl: S85-8

Han J. Y., et al. (2001) Enrichment and detection of fetal erythroid cells from maternal peripheral blood using liquid culture. Prenat Diagn. 21:22-6

Herman A., et al. (2000) Combined first trimester nuchal translucency and second trimester biochemical screening tests among normal pregnancies. Prenat Diagn. 20:781-4

Huber K., et al. (2000) Quantitative FISH analysis and in vitro suspension cultures of erythroid cells from maternal peripheral blood for the isolation of fetal cells. Prenat Diagn. 20:479-86

Huderer-Duric K., et al. (2000) The triple-marker test in predicting fetal aneuploidy: a compromise between sensitivity and specificity. Eur J Obstet Gynecol Reprod Biol. 88:49-55

Jansen M. W., et al. (2000) How useful is the in vitro expansion of fetal CD34+ progenitor cells from maternal blood samples for diagnostic purposes? Prenat Diagn. 20:725-31

Johnson J. W., et al. (1999) Technical factors in early amniocentesis predict adverse outcome. Results of the Canadian Early (EA) versus Mid-trimester (MA) Amniocentesis Trial. Prenat Diagn. 19:732-8

Lamvu G., et al. (1997) Prenatal diagnosis using fetal cells from the maternal circulation. Obstet Gynecol Surv. 52:433-7

Li R., et al. (1989) Identification of a human protein that interacts with nuclear localization signals. J Cell Biol. 109:2623-32

Lim T. H., et al. (2001) Relationship between gestational age and frequency of fetal trophoblasts and nucleated erythrocytes in maternal peripheral blood. Prenat. Diagn. 21:14-21

Lo Y. M. (2000) Fetal DNA in maternal plasma: biology and diagnostic applications. Clin Chem. 46:1903-6

Martel-Petit V., et al. (2001) Use of the Kleihauer test to detect fetal erythroblasts in the maternal circulation. Prenat Diagn 21:106-11

McDuffie R. S. Jr., et al. (1996) Prenatal screening using maternal serum alpha-fetoprotein, human chorionic gonadotropin, and unconjugated estriol: two year experience in a health maintenance organization. J Matern Fetal Med. 5:70-3

Rodriguez De Alba M., et al. (2001) Prenatal diagnosis on fetal cells from maternal blood: practical comparative evaluation of the first and second trimesters. Prenat Diagn. 21:165-70

Samura O., et al. (2000) Comparison of fetal cell recovery from maternal blood using a high density gradient for the initial separation step: 1.090 versus 1.119 g/ml. Prenat Diagn. 20:281-6

Sekizawa A., et al. (2000) Apoptosis in fetal nucleated erythrocytes circulating in maternal . . . Prenat Diagn. 20:886-9

Shulman L. P., et al. (1998) Frequency of nucleated red blood cells in maternal blood during the different gestational ages. Hum Genet. 103:723-6

Smith-Bindman R., et al. (2001) Second-trimester ultrasound to detect fetuses with Down syndrome: a meta-analysis. JAMA. 285:1044-55

Smits G., et al., (2000) An examination of different Percoll density gradients and magnetic activated cell sorting (MACS) for the enrichment of erythroblasts from maternal blood. Arch Gynecol Obstet. 263:160-3

Spencer K. (1999) Second trimester prenatal screening for Down's syndrome using alpha-fetoprotein and free beta hCG: a seven year review. Br J Obstet Gynaecol. 6:1287-93

Sundberg K., et al. (1997) Randomised study of risk of fetal loss related to early amniocentesis versus chorionic villus sampling. Lancet. 350:697-703

Tanski S., et al. (1999) Predictive value of the triple screening test for the phenotype of Down syndrome. Am J Med Genet. 85:123-6

Tutschek B., et al. (2000) Clonal culture of fetal cells from maternal blood. Lancet. 356:1736-7

Wang J. Y., et al. (2000) Fetal nucleated erythrocyte recovery: fluorescence activated cell sorting-based positive selection using anti-gamma globin versus magnetic activated cell sorting using anti-CD45 depletion and anti-gamma globin positive selection. Cytometry. 39:224-30

Weinans M. J., et al. (2000) How women deal with the results of serum screening for Down syndrome in the second trimester of pregnancy. Prenat Diagn. 20:705-8

Wilson R. D. (2000) Amniocentesis and chorionic villus sampling. Curr Opin Obstet Gynecol. 12:81-86

Wolf E. A., et al. (2000) Triple marker screening and pregnancy outcomes: statistical methods and results. Obstet Gynecol. 95:S43

Zhong X. Y., et al. (2000) Fetal DNA in maternal plasma is elevated in pregnancies with aneuploid fetuses. Prenat Diagn. 20:795-8

What is claimed is:

1. A method for selecting and identifying fetal erythroid cells comprising:
a. harvesting mononuclear cells from a blood sample;
b. adsorbing erythroid cells from the mononuclear cells onto a substrate;
c. applying molecular probes to differentiate maternal cells from fetal cells.

2. The method according to claim 1 further comprising using the differentially detected fetal cells for DNA-based prenatal diagnosis.

3. The method according to claim 1 wherein the blood sample is maternal peripheral blood.

4. The method according to claim 1 wherein the molecular probe is selected from the group consisting of antibodies against a fetal hemoglobin, oligonucleotide probes complementary to a fetal hemoglobin RNA, and molecular beacons complementary to a fetal hemoglobin RNA.

5. The method according to claim 1 wherein the substrate is a microscope slide coated with a coating that binds a protein present on the surface of fetal cells.

6. The method according to claim 5 wherein the coating comprises a layer of erythrocyte membranes bound to the surface of the slide and activated by treatment with antibodies that react with an erythroid cell surface protein.

7. The method according to claim 6 wherein the cell surface proteins are selected from the group consisting of glycophorin A, glycophorin B, and transferrin receptors.

8. The method according to claim 6 wherein the antibodies are directly attached chemically to the slide.

9. The method according to claim 6 wherein the antibodies are attached using S. aureus protein A.

10. A noninvasive method for detecting aneuploidies comprising:
a. harvesting mononuclear cells from a blood sample;
b. adsorbing erythroid cells from the mononuclear cells onto a substrate;
c. subjecting the cells attached to the substrate to fluorescence in situ hybridization;
d. applying at least one molecular beacon to the cells attached to the substrate so as to differentiate maternal cells from fetal cells.

11. The method according to claim 10 wherein the aneuploidy is Down syndrome.

12. The method according to claim 10 wherein the aneuploidies are selected from the group consisting of trisomy 13, trisomy 18, Klinefelter syndrome, XYY, and Turner syndrome.

13. The method according to claim 10 wherein the sex of a fetus is determined.

14. The method according to claim 1 wherein two molecular beacons are applied to the cells attached to the substrate.

15. The method according to claim 14 wherein a first molecular beacon is specific for gamma, zeta, or epsilon globin mRNA and a second molecular beacon is specific for beta globin mRNA.

16. The method according to claim 15 wherein the firs molecular beacon is labeled with rhodamine and the second molecular beacon is labeled with fluoresceine.

17. The method according to claim 10 wherein the molecular beacon is specific for gamma, zeta, or epsilon globin mRNA.

18. A method for detecting heritable conditions other than aneuploidies comprising:
a. adding to a blood sample containing maternal cells and fetal cells a permeabilizing detergent and at least one labeled molecular beacon;
b. sorting fetal cells from maternal cells in the sample;
c. analyzing fetal cells in the sample by PCR amplification.

19. The method according to claim 18 wherein two labeled molecular beacons are used.

20. The method according to claim 19 wherein a first molecular beacon is specific for gamma, zeta, or epsilon globin mRNA and a second molecular beacon is specific for beta globin mRNA.

21. The method according to claim 20 wherein the first molecular beacon is labeled with rhodamine and the second molecular beacon is labeled with fluoresceine.

22. The method according to claim 18 wherein the cells are sorted by FACS.

23. The method according to claim 22 wherein, prior to sorting the cells by FACS, the cells are enriched by removal of CD45-positive cells.

24. A method for enriching target cells in a sample comprising:
a. providing a substrate coated with a layer or erythrocyte membranes of the cells of interest;
b. activating the membranes of the cells of interest with antibodies that preferentially recognize cell surface proteins of the cells of interest;
c. adding a solution of cells to the substrate and allowing the cells to settle and attach to the coated substrate; and
d. removing nonabsorbed cells.

25. The method according to claim 24 wherein, after the nonabsorbed cells are removed, the cells of interest are permanently fixed to the substrate.

26. The method according to claim 24 wherein the antibodies are antibodies that react with an erythroid cell surface protein.

27. The method according to claim 26 wherein the cell surface proteins are selected from the group consisting of glycophorin A, glycophorin, B, and transferring receptors.

28. The method according to claim 24 wherein the cells of interest are fetal erythroid cells.
